(19) United States
(12) Patent Application Publication
(10) Pub. No.: US 2026/0168949 A1
(43) Pub. Date: Jun. 18, 2026

(54) SENSITIVE MEMBRANE AND GAS SENSOR

(71) Applicant: PANASONIC HOUSING SOLUTIONS CO., LTD., Osaka (JP)

(72) Inventors: Yosuke HANAI, Osaka (JP); Atsuo NAKAO, Nara (JP); Kazuki NAGASHIMA, Hokkaido (JP); Takeshi YANAGIDA, Tokyo (JP)

(21) Appl. No.: 18/685,822

(22) PCT Filed: Aug. 24, 2022
(86) PCT No.: PCT/JP2022/031922

§ 371 Date: Mar. 5, 2026

(30) Foreign Application Priority Data
Aug. 27, 2021 (JP) 2021-139306

(51) Int. Cl. G01N 27/12; G01N 33/00

(52) U.S. Cl. CPC G01N 27/126; G01N 33/0027

(57) ABSTRACT

Provided is a sensitive membrane, of which the membrane body is less likely to cause deterioration in its performance. A sensitive membrane 20 includes: a membrane body 201 containing a sensitive material; a conductive material contained in the membrane body 201; and an oxidation inhibitor. The oxidation inhibitor is contained in the membrane body 201. The oxidation inhibitor inhibits oxidation of the sensitive material.

### TECHNICAL FIELD

The present disclosure generally relates to a sensitive membrane and a gas sensor, and more particularly relates to a sensitive membrane including an oxidation inhibitor and a gas sensor that uses such a sensitive membrane.

### BACKGROUND ART

Patent Literature 1 discloses a resin composition for use in an odor identification probe. The resin composition includes a resin, a surfactant, and a conductive carbon material. Patent Literature 1 also discloses an odor identification sensor detector containing such a resin composition for use in an odor identification probe. Patent Literature 1 further discloses a detector array, including two or more such detectors, for use in an odor identification sensor and further discloses an identification sensor including such a detector array for use in an odor identification sensor

In the sensor of Patent Literature 1, however, the resin (organic polymer) included in the resin composition for use in an odor identification probe may deteriorate depending on the measuring environment. Thus, it is difficult for the sensor of Patent Literature 1 to provide stabilized sensor output for a long term.

### CITATION LIST

### Patent Literature

Patent Literature 1: JP 2021-032842 A

### SUMMARY OF INVENTION

An object of the present disclosure is to provide a sensitive membrane which is less likely to deteriorate with time.

Another object of the present disclosure is to provide a gas sensor with sensor sensitivity that is less likely to decrease.

A sensitive membrane according to an aspect of the present disclosure includes: a membrane body containing a sensitive material; a conductive material contained in the membrane body; and an oxidation inhibitor. The oxidation inhibitor is contained in the membrane body. The oxidation inhibitor inhibits oxidation of the sensitive material.

A gas sensor according to another aspect of the present disclosure includes: the sensitive membrane described above; and an electrode electrically connected to the conductive material.

### BRIEF DESCRIPTION OF DRAWINGS

FIG. 1A is a plan view illustrating a gas sensor according to an exemplary embodiment;

FIG. 2B is a perspective view illustrating a sensitive membrane of the gas sensor according to the exemplary embodiment;

FIGS. 2A and 2B illustrate how the sensitive membrane operates; FIG. 2C is a graph showing, as an example, how the resistance value may change with time through the operation of the sensitive membrane;

FIG. 3 is a graph showing relationships between the sensitivity change rate and the number of days of an accelerated aging test in first to third examples of the exemplary embodiment and in a first comparative example;

FIG. 4 is a graph showing a relationship between the initial sensitivity and the concentration of ascorbic acid in the first to third examples of the exemplary embodiment and in the first comparative example;

FIG. 5 is a graph showing how the sensitivity changed before and after the accelerated aging test in fourth to sixth examples of the exemplary embodiment and in a second comparative example;

FIG. 6 is a graph showing how the resistance changed before and after the accelerated aging test in the fourth to sixth examples of the exemplary embodiment and in the second comparative example; and

FIG. 7 is a graph showing relationships between the sensitivity change rate and the number of days of storage in a seventh example of the exemplary embodiment and in a third comparative example.

### DESCRIPTION OF EMBODIMENTS

### First Embodiment

### (1) Overview

A gas sensor 1 according to an exemplary embodiment of the present disclosure may be, for example, an artificial olfactory sensor and may be used to, for example, detect odor molecules (i.e., a substance that stimulates the human olfactory sensation) as detection target gas molecules. Examples of the odor molecules include volatile organic compounds (VOCs) and ammonia. The gas sensor 1 may be used to detect VOCs, for example. The gas sensor 1 detects VOCs as odor gas molecules included in a sample gas such as a gas taken from a food, a breath taken from a human body, or the air taken from a building room. Note that the detection target gas molecules to be detected by the gas sensor 1 do not have to be VOCs but may also be multiple types of odor molecules including VOCs or non-odor molecules such as molecules of a flammable gas or a poisonous gas like carbon monoxide.

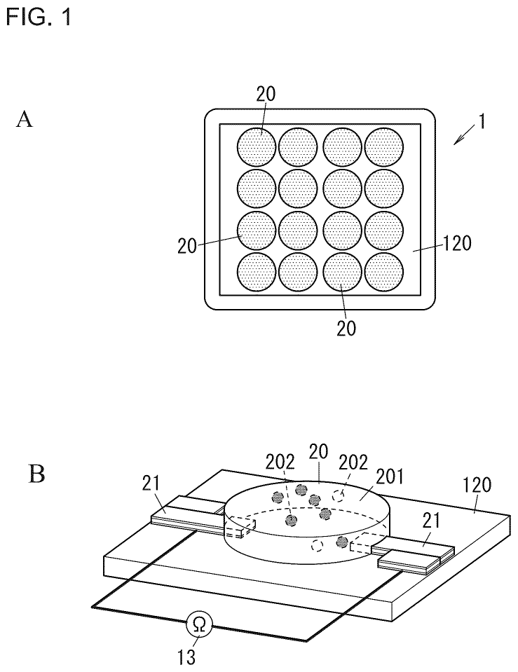

FIG. 1A illustrates a gas sensor 1 according to this embodiment. This gas sensor 1 includes, on a substrate 120, at least one sensitive membrane 20 and a plurality of electrodes 21. This gas sensor 1 includes a plurality of sensitive membranes 20. With respect to each of these sensitive membranes 20, a plurality of (e.g., a pair of) electrodes 21 are arranged to interpose the sensitive membrane 20 between themselves. A number of sensitive membranes 20 are arranged vertically and horizontally to form an array (e.g., a 4×4 array in this embodiment) of sensitive membranes 20. Each of these sensitive membranes 20 is formed in a circular pattern when viewed in plan. Note that the number, arrangement, and shape of the sensitive membranes 20 in the gas sensor 1 do not have to be the ones shown in FIG. 1A but may also be changed as appropriate according to the type of the gas sensor 1, for example. It should also be noted that the number, arrangement, shape, and material of the electrodes 21 in the gas sensor 1 do not have to be the ones shown in FIG. 1A but may also be changed as appropriate according to the type of the gas sensor 1, for example.

The sensitive membrane 20 according to this embodiment includes: a membrane body 201 containing a sensitive material; a conductive material; and an oxidation inhibitor. That is to say, the sensitive membrane 20 is a composite membrane made of multiple materials including the sensitive material, the conductive material, and the oxidation inhibitor.

As shown in FIG. 1B, the conductive material is formed to include a plurality of conductive particles 202. The sensitive membrane 20 is formed by dispersing the plurality of conductive particles 202 in the membrane body 201. Each electrode 21 is electrically connected to the conductive particles 202 in the membrane body 201. In addition, the pair of electrodes 21 are also electrically connected to a detection unit in a processing unit 13.

The membrane body 201 is formed to be ready to adsorb gas molecules G as detection target molecules. The membrane body 201 also has electrical insulation properties and is formed out of the sensitive material in the shape of a membrane, a plate, or a sheet. The sensitive material as a constituent material for the membrane body 201 includes either an organic polymer or an ionic liquid. An appropriate type of organic polymer or ionic liquid is selected according to, for example, the type of a chemical substance (gas) to be adsorbed by the membrane body 201 and the type of the conductive particles 202.

The conductive particles 202 are particles with electrical conductivity. The sensitive membrane 20 is made electrically conductive by including the plurality of conductive particles 202. The conductive particles 202 may include at least one material selected from the group consisting of, for example, carbon materials, conductive polymers, metals, metal oxides, semiconductors, superconductors, and complex compounds.

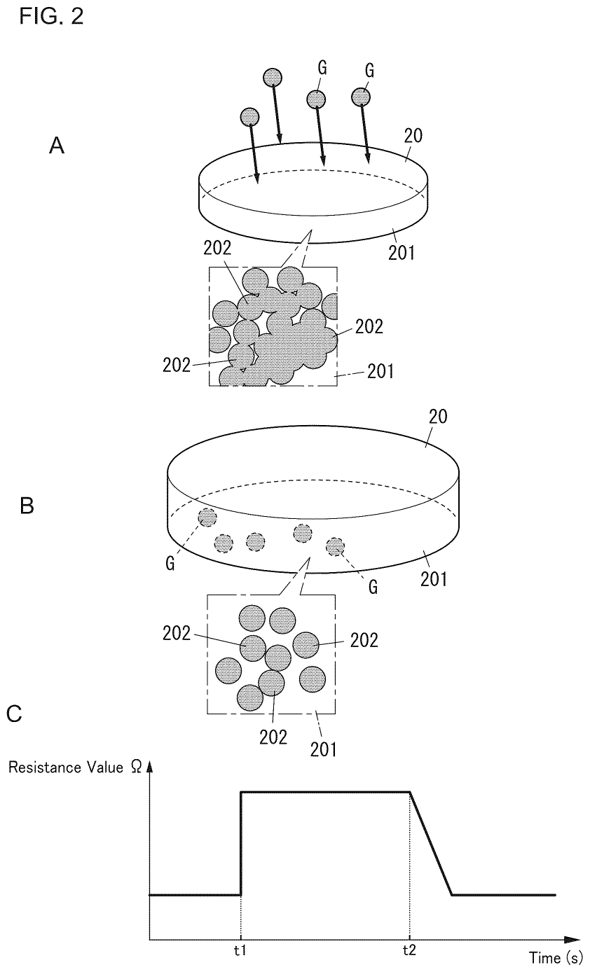

In such a sensitive membrane 20, the membrane body 201 is less thick before adsorbing the gas molecules G as shown in FIG. 2A. That is to say, the plurality of conductive particles 202 are dispersed more densely in the membrane body 201. Thus, the resistance value of the sensitive membrane 20 as detected by the processing unit 13 is smaller than in a situation where the gas molecules G are adsorbed into the membrane body 201.

Once the sensitive membrane 20 has adsorbed the gas molecules G from the state shown in FIG. 2A, the membrane body 201 expands in response of adsorption of the gas molecules G to have an increased thickness. That is to say, the plurality of conductive particles 202 are dispersed more sparsely in the membrane body 201 as shown in FIG. 2B. As a result, the interval between the plurality of conductive particles 202 dispersed in the membrane body 201 broadens, thus making the resistance value when the sensitive membrane 20 adsorbs the gas molecules G at a time t1 larger than the resistance value before the sensitive membrane 20 adsorbs the gas molecules G as shown in FIG. 2C. Meanwhile, as the gas molecules G desorb from the sensitive membrane 20, the membrane body 201 shrinks to have a decreased thickness (i.e., the state shown in FIG. 2A) from the thickened state (i.e., the state shown in FIG. 2B). As a result, the resistance value of the sensitive membrane 20 gradually decreases since a time 12 when the gas molecules G start to desorb. The gas sensor 1 may determine, by making the detection unit of the processing unit 13, which is electrically connected to the electrodes 21, detect this change in resistance value, whether there are any gas molecules G in the gas such as the air supplied to the gas sensor 1.

In addition, the sensitive membrane 20 according to this embodiment includes an oxidation inhibitor in the membrane body 201 containing the sensitive material. This allows the oxidation of the sensitive material to be inhibited by the oxidation inhibitor, thus making the sensitive material less easily decomposable due to oxidation. Consequently, this reduces the chances of causing a decrease in the sensitivity performance of the membrane body 201. That is to say, the functions proper to the membrane body 201, which expands and comes to have an increased volume when adsorbing the gas molecules G and which shrinks and comes to have a decreased volume when desorbing the gas molecules G, are less likely to be lost with time. Consequently, this also reduces the chances of the sensitive membrane 20 and the gas sensor 1 losing their proper functions, thus making it easier for the gas sensor 1 to maintain its sensor sensitivity for a long term.

### (2) Details

### (2.1) Configuration

### <Sensitive Membrane and Gas Sensor>

The gas sensor 1 according to this embodiment includes the sensitive membrane 20 and the plurality of electrodes 21 as shown in FIG. 1A.

Also, the sensitive membrane 20 according to this embodiment includes: the membrane body 201 containing the sensitive material; the conductive material; and the oxidation inhibitor.

### <<Membrane Body>>

The sensitive material contained in the membrane body 201 is a material having a lower degree of electrical conductivity than the conductive material and having electrical insulation properties. The sensitive material includes either an organic polymer or an ionic liquid.

Preferred examples of the organic polymer include materials commercially available as stationary phases for columns in gas chromatographs. More specifically, the organic polymer may include at least one material selected from the group consisting of, for example, polyethers such as polyalkylene glycols, polyesters, silicones, glycerols, nitriles, dicarboxylic acid monoesters, and aliphatic amines. In this case, the membrane body 201 may easily adsorb chemical substances, especially volatile organic compounds, in the gas.

The polyalkylene glycols may include, for example, polyethylene glycol (with a heat resistant temperature of 170° C.). The polyesters may include, for example, at least one material selected from the group consisting of poly(diethylene glycol adipate) and poly(ethylene succinate). The silicones may include, for example, at least one material selected from the group consisting of dimethyl silicone, phenyl methyl silicone, trifluoropropyl methyl silicone, and cyano-silicone (with a heat resistant temperature of 275° C.). The glycerols may include, for example, diglycerol (with a heat resistant temperature of 150° C.). The nitriles may include, for example, at least one material selected from the group consisting of N,N-bis(2-cyanoethyl) formamide (with a heat resistant temperature of 125° C.) and 1,2,3-tris(2-cyanoethoxy) propane (with a heat resistant temperature of 150° C.). The dicarboxylic acid monoesters may include, for example, at least one material selected from the group consisting of nitroterephthalic acid-modified polyethylene glycol (with a heat resistant temperature of 275° C.) and diethylene glycol succinate (with a heat resistant temperature of 225° C.). The aliphatic amines may include, for example, tetrahydroxyethyl ethylenediamine (with a heat resistant temperature of 125° C.).

The ionic liquid is a salt (low molecular substance) which is liquid at an ordinary temperature and causes less steric hindrances than a high molecular substance that has been used for a sensitive membrane of a gas sensor. That is why the gas molecules G as detection target molecules would be adsorbed into the membrane body 201 easily and the gas molecules G adsorbed into the membrane body 201 would have a high diffuse rate in the membrane body 201. Consequently, this causes an increase in the response speed of the gas sensor 1. In addition, the membrane body 201 containing the ionic liquid also desorbs the gas molecules G at high speeds. Thus, the gas sensor 1 according to this embodiment may cause a significant structural change reversibly to the conductive particles by making the ionic liquid as the gas adsorbent of the sensitive membrane 20 adsorb and desorb the gas molecules G at high speeds.

Besides, the ionic liquid has so low a vapor pressure as to vaporize hardly. This makes it easier to maintain the shape of the sensitive membrane 20. Furthermore, the ionic liquid has so high stability that the chemical structure thereof changes less significantly and hardly deteriorates. Moreover, the ionic liquid may have its properties changed when modified by any of various combinations of cations and anions or respective cations or anions. Thus, multiple different types of ionic liquids may be formed in theory by 1016 different combinations of cations and anions. Therefore, if a plurality of membrane bodies 201 are configured as respective combinations of multiple different types of cations and anions, it makes it easier for the plurality of membrane bodies 201 to adsorb multiple different types of gas molecules G, which is advantageous to provide a gas sensor 1 with multi-channel capability. That is to say, this increases the selectivity of a desired type of gas molecules G as the detection target molecules for the gas sensor 1, thus enabling the type of the gas molecules G to be identified more accurately.

In this embodiment, examples of cation (species) of the ionic liquid include imidazolium (5-membered ring, conjugated), piperidinium (6-membered ring, single bond), pyrrolidinium (5-membered ring, single bond), pyridinium (6-membered ring, conjugated), ammonium, sulfonium, and phosphonium. In this embodiment, examples of anion (species) of the ionic liquid include a carboxylate ion, a phosphate ion, a sulfonate ion, a tetrafluoroboronate ion, a trifluoromethyl group ([Tf₂N]⁻, hydrophobic), a hexafluorophosphate ion, and trifluoromethanesulfonate ([TfO]⁻, hydrophobic).

In this embodiment, the anion of the ionic liquid is preferably a hydrophobic anion. This reduces the chances of moisture being adsorbed into the membrane body 201 of the sensitive membrane 20, thus increasing the sensitivity of the gas sensor 1 to the gas molecules G as the detection target molecules. That is to say, the air contains not only the gas molecules G but also a lot of water molecules (moisture) as well. The water molecules have a far higher concentration than the gas molecules G, and therefore, a plenty of water molecules are easily adsorbed into the membrane body 201. That is why the moisture affects the detection result of the gas sensor 1 so significantly that it is difficult for gas sensor 1 to have good response to the gas molecules G as the detection target molecules. To overcome this problem, according to this embodiment, a hydrophobic anion is used as the ionic liquid of the membrane body 201, thus reducing the chances of water molecules being adsorbed into the membrane body 201 and thereby reducing the effect of the moisture on the detection result of the gas sensor 1.

As used herein, to be “hydrophobic” would be substantially synonymous with having low hydrogen bond acceptability. Since the reactivity between water and the ionic liquid heavily depends on a hydrogen bond, the reactivity would be reduced by using an anion with a low degree of hydrogen bond acceptability as the anion of the ionic liquid. In that case, —OH produced by polarization of water is a hydrogen bond donor and N, O, F, and other atoms produced by polarization of the anion are hydrogen bond acceptors. The hydrophobic anion preferably has a hydrogen bond acceptability parameter (β value) less than 0.3, for example. The smaller the β value is, the less likely the anion forms a hydrogen bond to water. The lower limit of the β value is not set at any particular value but only needs to be greater than zero.

As the hydrophobic anion, an organic fluorine compound is preferably used. This decreases the hydrogen bond acceptability of the hydrophobic anion, thus reducing the chances of the moisture being adsorbed into the membrane body 201. Also, the organic fluorine compound for use as the hydrophobic anion is preferably a compound having a trifluoromethyl group. This further decreases the hydrogen bond acceptability of the hydrophobic anion, thus further reducing the chances of the moisture being adsorbed into the membrane body 201. Specific examples of such a compound having a trifluoromethyl group include bis(trifluoromethanesulfonyl)amide ion (see the following chemical formula (1)). Note that the hydrophobic anion preferably has no carboxy groups. This makes it easier for the hydrophobic anion to exhibit hydrophobicity.

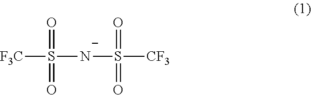 [SMILES: O=S(=O)([N-]S(=O)(=O)C(F)(F)F)C(F)(F)F]

In this embodiment, imidazolium is preferably used as the cation of the ionic liquid. Moreover, it is preferable to use a highly hydrophobic cation such as imidazolium having an alkyl chain with seven or more carbon atoms. The imidazolium for use in this embodiment is expressed by the following chemical formula (2):

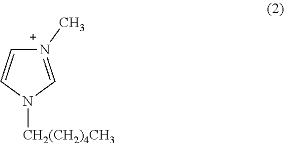

The ionic liquid that forms the membrane body 201 may contain cations and anions at a constant ratio. For example, the ionic liquid may contain monovalent anions and cations at an equal ratio from the viewpoint of valence.

### <<Conductive Material>>

The conductive material contained in the membrane body 201 is a material having a higher degree of conductivity than the membrane body 201. The conductive material is made up of a plurality of conductive particles 202. The plurality of conductive particles 202 are dispersed uniformly in the membrane body 201. As used herein, the term “uniformly” does not necessarily mean “perfectly uniformly” in a strict sense but may also mean “almost uniformly.”

The conductive material includes at least one selected from the group consisting of carbon blacks, carbon nanotubes, metallic nanoparticles, and conductive polymers. Among other things, a carbon black is preferably used as the conductive material to allow the gas sensor 1 to have high sensitivity. This particularly significantly increases the chances of the sensitive membrane 20 causing a change in its electrical resistance value when the gas sensor 1 is exposed to a gas.

In general, there are two types of carbon blacks, namely, a “conductive carbon black” and a “coloring carbon black.” The conductive carbon black is mainly used as a conductive material in various fields for films, IC trays, sheet heating elements, magnetic tapes, and conductive rubber. The coloring carbon black is mainly used as a black pigment in various fields for newspaper inks, printing inks, resin coloring, paints, and toners. The conductive carbon black and the coloring carbon black may be distinguished by the degree of development of a network structure (i.e., so-called “structure”) formed by carbon black particles (conductive particles 202). The conductive carbon black has a well-developed structure, while the coloring carbon black has a structure which is developed less fully than the conductive carbon black. That is to say, the structure is formed by bonding carbon black particles together both chemically and physically. The carbon black with the well-developed structure has a larger number of carbon black particles that are chemically and physically bonded together. On the other hand, the carbon black with an undeveloped structure has a smaller number of carbon black particles that are bonded together chemically and physically.

In this embodiment, a carbon black with an undeveloped structure is preferably used as the carbon black. Specifically, in this embodiment, a carbon black having a dibutyl phthalate absorption number (hereinafter referred to as a “DBP absorption number”) less than 100 cm³/100 g is preferably used as the carbon black. Meanwhile, a carbon black having a DBP absorption number equal to or greater than 100 cm³/100 g has a well-developed structure, and therefore, is preferably not used in this embodiment. Note that the DBP absorption number herein refers to the number of DBP (dibutyl phthalate) particles absorbed into 100 g of carbon black and is measured in accordance with the JIS K 6221 standard.

As for the mechanism that causes a carbon black to have electrical conduction in the membrane body 201, there are two competitive theories, namely, a so-called “conductive passage theory,” according to which π electrons move through the structure, and a so-called “tunneling effect theory,” according to which electrical conduction is produced by causing π electrons to jump through the gap between the particles. The carbon black having a DBP absorption number equal to or greater than 100 cm³/100 g has such a developed structure that the electrical conduction through the conductive passage would be prevailing in the carbon black of this type. On the other hand, the carbon black having a DBP absorption number less than 100 cm³/100 g has such an undeveloped structure that the electrical conduction due to the tunneling effect would be prevailing in the carbon black of this type. In the sensitive membrane 20 according to this embodiment, the electrical conduction would be produced by the tunneling effect between the carbon black particles, thus causing the resistance value to change more significantly due to adsorption of the gas molecules (odor molecules) G and thereby allowing the gas sensor 1 to have higher sensitivity.

Note that the material for the metallic nanoparticles does not have to be made of a single metallic element but may also be made of, for example, a metal oxide, a semiconductor, a superconductor, or a complex compound. For example, the conductive particles 202 preferably include an oxide semiconductor, which is preferably antimony tin oxide. This particularly significantly increases the chances of the sensitive membrane 20 causing a change in its electrical resistance value when the gas sensor 1 is exposed to a gas.

The conductive particles 202 preferably have a mean particle size equal to or greater than 10 nm and equal to or less than 300 nm, for example. This may cause an increase in the dispersibility of the conductive particles 202 in the membrane body 201. Note that the mean particle size of the conductive particles 202 is a number-based arithmetic mean of particle sizes which is calculated using an electron micrograph of the conductive particles 202.

The ratio of the conductive material contained in the sensitive membrane 20 is not limited to any particular value. For example, the ratio of the conductive material is preferably 200 parts by mass with respect to 100 parts by mass of the membrane body 201. This particularly significantly increases the chances of the sensitive membrane 20 causing a change in its electrical resistance value when the gas sensor 1 is exposed to a gas.

### <<Oxidation Inhibitor>>

The oxidation inhibitor contained in the membrane body 201 has the function of inhibiting the oxidation of the sensitive material. That is to say, oxidation of the oxidation inhibitor itself reduces the action of oxygen on the sensitive material, thus making the sensitive material less easily oxidizable. The oxidation inhibitor includes at least one selected from the group consisting of aromatic compounds, sulfur compounds, phosphorus compounds, amine compounds, metallic compounds, vitamin E, and vitamin C.

As the aromatic compound, at least one compound selected from the group consisting of: 2,2′-methylenebis(6-cyclohexyl-p-cresol), 4,6-di-tert-butylresorcinol, 2-methyl-4,6-bis[(n-octylthio) methyl]phenol, 2,4-bis[(dodecylthio)methyl]-6-methylphenol, 2,2′-methylene-bis(4-methyl-6-tert-butylphenol), 2-(1,1-dimethylethyl)-4-methoxy-phenol, 2,6-di-tert-butyl-p-cresol, 2,2′,6,6′-tetra-tert-butyl-4,4′-dihydroxybiphenyl, 2,6-di-tert-butylphenol, 4-(hexyloxy)-2,3,6-trimethylphenol, 3,6-dihydroxybenzonorbornane, 2,4,6-tris(3′,5′-di-tert-butyl-4′-hydroxybenzyl) mesitylene, 4,4′,4″-(1-methylpropanyl-3-ylidene)tris(6-tert-butyl-m-cresol), 6-tert-butyl-2,4-xylenol, 3-(3,5-di-tert-butyl-4-hydroxyphenyl) propionate, stearyl 3-(3,5-di-tert-butyl-4-hydroxyphenyl) propionate, 2,4,6-tris(2,4-dihydroxyphenyl)-1,3,5-triazine, N,N′-bis-3-(3,5-di-tert-butyl-4-hydroxyphenyl) propionylhexamethylenediamine, galvinoxyl free radical, 4,4′-dihydroxy-3,3′,5,5′-tetraisopropylbiphenyl, (1,1-dimethylethyl)-4-methoxy-phenol, 1,3,5-tris (3,5-di-tert-butyl-4-hydroxybenzyl)-1,3,5-triazinane-2,4,6-trione, 3-(3,5-di-tert-butyl-4-hydroxy-phenyl)-N′-[3-(3,5-di-tert-butyl-4-hydroxyphenyl) propanoyl]propanehydrazide, 2,6-di-tert-butyl-4-ethylphenol, 2,6-di-tert-butyl-4-methoxyphenol, 2-tert-butyl-6-(3-tert-butyl-2-hydroxy-5-methylbenzyl)-4-methylphenylacrylate, styrenated phenol, 3-(3,5-di-tert-butyl-4-hydroxyphenyl)methylpropionate, pentaerythritol tetrakis[3-(3,5-di-tert-butyl-4-hydroxyphenyl) propionate], hexadecyl 3,5-di-tert-butyl-4-hydroxybenzoate, N,N′-bis{02-[2-(3,5-di-tert-butyl-4-hydroxy-phenyl)ethylcarbonyloxy]ethyl>oxamide, 2,2′-methylenebis[6-(1-methylcyclohexyl)-p-cresol], 2,5-di-tert-amylhydroquinone, 3,3′,5,5′-tetra-tert-butyl-4,4′-stilbenequinone, 4,4′-butylidene-bis(6-tert-butyl-m-cresol), 2,2′-methylene-bis(4-ethyl-6-tert-butylphenol), 3-(1,1-dimethylethyl)-4-methoxy-phenol, 2,5-di-tert-butylhydroquinone, 2,5-bis(1,1,3,3-tetramethylbutyl) hydroquinone, 2,4,8,10-tetraoxaspiro[5.5]undecane-3,9-diylbis(2-methylpropane-2,1-diyl)bis[3-[3-(tert-butyl)-4-hydroxy-5-methylphenyl]propanoate], 4,4′-thiobis(6-tert-butyl-m-cresol), diethyl 3,5-di-tert-butyl-4-hydroxybenzylphosphonate, and 4-[[4,6-bis(n-octylthio)-1,3,5-triazine-2-yl]amino]-2,6-di-tert-butylphenol may be used.

As the sulfur compound, at least one selected from the group consisting of di(tridecyl) 3,3′-thiodipropionate, didodecyl 3,3′-thiodipropionate, nickel (II) dibutyldithiocarbamate, nickel diethyldithiocarbamate, 2,2-bis {[3-(dodecylthio)-1-oxopropoxy]methyl}propane-1,3-diylbis[3-(dodecylthio) propionate], and 2-mercaptobenzimidazole may be used.

As the phosphorus compound, at least one selected from the group consisting of tritolyl phosphite, triphenyl phosphite, tributyl phosphite, 2,2′-methylenebis(4,6-di-tert-butylphenyl) 2-ethylhexyl phosphite, so-called “Trioleyl phosphite,” 2-ethylhexyl diphenyl phosphite, triisodecyl phosphite, tris(nonylphenyl) phosphite, isodecyldiphenyl phosphite, 3,9-bis(2,4-di-tert-butylphenoxy)-2,4,8,10-tetraoxa-3,9-diphosphaspiro[5.5]undecane, tris(2-ethylhexyl)phosphite, trioctyl phosphite (mixture), tris(2,4-di-tert-butylphenyl) phosphite, 3,9-bis(octadecyloxy)-2,4,8,10-tetraoxa-3,9-diphosphaspiro[5.5]undecane, trihexyl phosphite, tri-p-tolyl phosphite, tris(1,1,1,3,3,3-hexafluoro-2-propyl)phosphite, 3,9-bis(2,6-di-tert-butyl-4-methylphenoxy)-2,4,8,10-tetraoxa-3,9-diphosphaspiro[5.5]undecane, and tetra-C12-15-alkyl(propane-2,2-diylbis (4,1-phenylene))bis(phosphite) may be used.

As the amine compound, at least one selected from the group consisting of 4,4′-bis(α,α-dimethylbenzyl)diphenylamine, 4-isopropylaminodiphenylamine, N,N′-di-sec-butyl-1,4-phenylenediamine, a 2,2,4-trimethyl-1,2-dihydroquinoline polymer, benzenamine, N-phenyl reaction product and 2,4,4-trimethylpentene, a diphenylamine derivative, N,N′-diphenyl-1,4-phenylenediamine, N-1,3-dimethylbutyl-N′-phenyl-p-phenylenediamine, N-phenyl-1-naphthylamine, 6-ethoxy-2,2,4-trimethyl-1,2-dihydroquinoline, and N,N′-di-2-naphthyl-1,4-phenylenediamine may be used.

As the metallic compound, at least one selected from the group consisting of dibutyltin maleate, zinc dibutyldithiocarbamate, zinc dimethyldithiocarbamate, nickel dibutyldithiocarbamate, zinc diethyldithiocarbamate, 2-mercaptobenzimidazole, and dibutyltin dilaurate may be used.

As the vitamin E, at least one selected from the group consisting of α-tocopherol, β-tocopherol, γ-tocopherol, and δ-tocopherol may be used.

As the vitamin C, at least one selected from the group consisting of L-ascorbic acid, sodium L-ascorbate, L-ascorbic stearate, L-ascorbic palmitate, L-ascorbic acid 2-glucoside, and isoascorbic acid may be used.

It is generally believed that when heat, light, catalyst, or any other form of stimulus causes an action in the presence of oxygen, carbon-hydrogen bonds, having relatively weak bonding strength, would be decoupled in a molecule of the sensitive material to produce free radicals with a high degree of reactivity and thereby advance degradation reaction as a chain reaction. They believe that oxidation inhibitors would inhibit oxidation by the following mechanism: oxidation inhibitor molecules, such as phenols and amines, in the oxidation inhibitors would react with, and deactivate, free radicals, thus bringing the degradation reaction to a halt.

That is why the same advantage would be achieved by introducing these oxidation inhibitor molecules into the molecular structure of the sensitive material.

In addition, in the sensitive membrane 20 according to this embodiment, the electrical conduction is produced by the tunneling effect between the carbon black particles. Thus, arranging the sensitive material and the oxidation inhibitor uniformly in the interface between the carbon particles in the sensitive membrane 20 would provide a gas sensor that is even less likely to cause a decrease in the sensor sensitivity.

### <Manufacturing of Gas Sensor>

The gas sensor 1 according to this embodiment is formed by providing a plurality of sensitive membranes 20 and a plurality of electrodes 21 on a substrate 120. A pair of electrodes 21 are in contact with each sensitive membrane 20 to electrically connect the conductive material in the sensitive membrane 20 to the plurality of electrodes 21. To manufacture the gas sensor 1, the plurality of sensitive membranes 20 are formed on the substrate 120 on which the plurality of electrodes 21 have been formed. Each sensitive membrane 20 may be formed by applying a molding material (nanocomposite material), containing a sensitive material, a conductive material, and an oxidation inhibitor, by a technique such as an inkjet method or a dispense method.

### (2.2) Advantages

In this embodiment, the sensitive membrane 20 contains an oxidation inhibitor in the membrane body 201 thereof. Thus, the oxidation inhibitor makes the sensitive material contained in the membrane body 201 less easily oxidable, thus reducing the chances of causing decomposition of the sensitive material due to oxidation. The following reaction scheme (1) shows a situation where the sensitive material is polyethylene glycol, and the oxidation inhibitor is ascorbic acid. If no ascorbic acid were contained in the membrane body 201, then an oxygen molecule would act on polyethylene glycol, thus often causing the polyethylene glycol to be decomposed by having its ester bond cut off, for example, and come to have a lower molecular weight. On the other hand, if ascorbic acid were contained in the membrane body 201, then the ascorbic acid would be easily oxidized, instead of polyethylene glycol, thus reducing the chances of oxygen molecules acting on the polyethylene glycol. This makes the polyethylene glycol less easily decomposable and makes it easier for the polyethylene glycol to maintain its initial high molecular state. That is why the gas sensor 1 according to this embodiment may reduce the chances of causing deterioration with time in the functions such as expansion and shrinkage proper to the membrane body 201 and eventually reduce the chances of causing a decline in the functions proper to the sensitive membrane 20 and the gas sensor 1. Consequently, this makes it easier for the gas sensor 1 to maintain its sensor sensitivity for a long term.

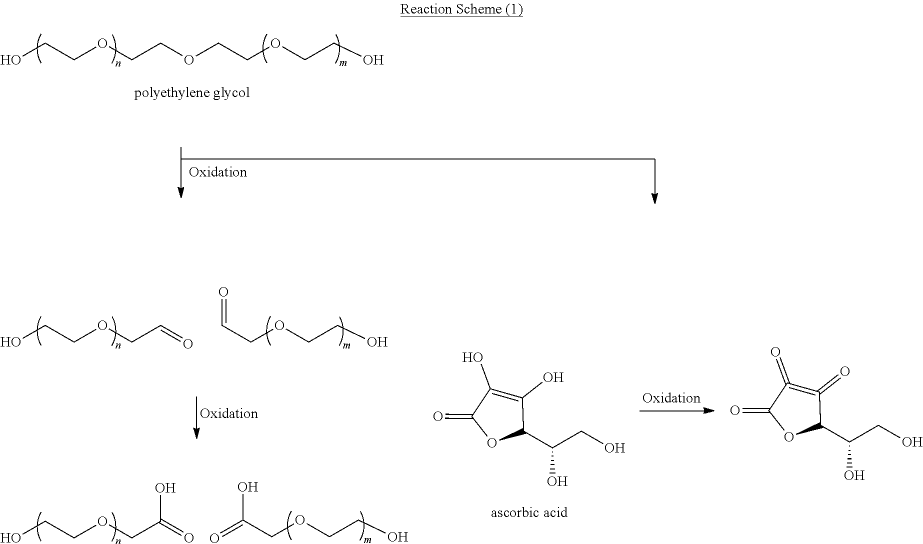

Furthermore, in the gas sensor 1, the sensor sensitivity thereof would deteriorate due to not only oxidation of the sensitive material but also adsorption of water molecules to the membrane body 201. According to this embodiment, however, even if water molecules are adsorbed into the membrane body 201 thereof, the gas sensor 1 may still have its sensor sensitivity recovered when subjected to a heat treatment within a dry atmosphere. That is to say, if oxygen is contained even a little in the dry atmosphere for the heat treatment, the sensitive material could be decomposed without an oxidation inhibitor in the sensitive membrane 20. On the other hand, according to this embodiment, the membrane body 201 contains the oxidation inhibitor. Thus, even if adsorption of water molecules once causes a decrease in the sensitivity of the sensor, the sensor may still have its sensitivity recovered easily and repeatedly.

In addition, the gas sensor 1 is formed to have a relatively small sensor size (such as the configuration according to this embodiment shown in FIG. 1A). Thus, the same advantage as the one produced by conducting a heat treatment in a dry atmosphere may be achieved due to the self-heating effect of the sensitive membrane 20 simply by applying a current to the sensor in the dry atmosphere.

### (Recapitulation)

As can be seen from the foregoing description, a sensitive membrane (20) according to a first aspect includes: a membrane body (201) containing a sensitive material; a conductive material contained in the membrane body (201); and an oxidation inhibitor. The oxidation inhibitor is contained in the membrane body (201) to inhibit oxidation of the sensitive material.

According to the first aspect, oxidation of the sensitive material in the membrane body (201) is inhibited by the oxidation inhibitor, thus making the sensitive material less easily decomposable. This reduces the chances of causing deterioration in the performance of the membrane body (201). Consequently, the sensor sensitivity is less likely to decrease when the sensitive membrane (20) is applied to a sensor such as a gas sensor.

In a sensitive membrane (20) according to a second aspect, which may be implemented in conjunction with the first aspect, the sensitive material includes an organic polymer.

The second aspect not only makes it easier to ensure electrical insulation properties and heat resistance for the membrane body (201) but also improves the ability of the membrane body (201) to adsorb volatile organic matter as well.

In a sensitive membrane (20) according to a third aspect, which may be implemented in conjunction with the first aspect, the sensitive material includes an ionic liquid.

The third aspect may accelerate adsorption of gas molecules into the membrane body (201) and desorption of gas molecules from the membrane body (201), thus making it easier to increase the response speed when the sensitive membrane (20) is applied to a gas sensor.

In a sensitive membrane (20) according to a fourth aspect, which may be implemented in conjunction with any one of the first to third aspects, the conductive material includes at least one selected from the group consisting of carbon blacks, carbon nanotubes, metallic nanoparticles, and conductive polymers.

The fourth aspect makes the conductive material dispersible more uniformly in the membrane body (201), thus making it easier to improve the sensor sensitivity.

In a sensitive membrane (20) according to a fifth aspect, which may be implemented in conjunction with any one of the second to fourth aspects, the organic polymer includes at least one selected from the group consisting of polyethers, polyesters, and silicones.

The fifth aspect makes it easier to ensure electrical insulation properties and heat resistance for the membrane body (201)

In a sensitive membrane (20) according to a sixth aspect, which may be implemented in conjunction with any one of the first to fifth aspects, the oxidation inhibitor includes at least one selected from the group consisting of aromatic compounds, sulfur compounds, phosphorus compounds, amine compounds, metallic compounds, vitamin E, and vitamin C.

The sixth aspect makes the sensitive material less easily oxidizable.

In a sensitive membrane (20) according to a seventh aspect, which may be implemented in conjunction with any one of the first to sixth aspects, content of the oxidation inhibitor is equal to or greater than 10% by mass and equal to or less than 50% by mass with respect to the sensitive material.

The seventh aspect makes the sensitive material less easily oxidizable.

A gas sensor (1) according to an eighth aspect includes: the sensitive membrane (20) according to any one of the first to seventh aspects; and an electrode (21) electrically connected to the conductive material.

According to the eighth aspect, oxidation of the sensitive material in the membrane body (201) is inhibited by the oxidation inhibitor, thus making the sensitive material less easily decomposable. This reduces the chances of causing deterioration in the performance of the membrane body (201). Consequently, the sensor sensitivity is less likely to decrease when the sensitive membrane (20) is applied to the gas sensor (1).

### EXAMPLES

### First to Third Examples and First Comparative Example

In first to third examples and a first comparative example, a gas sensor, including a sensitive membrane made of a polymer-carbon black nanocomposite, was formed.

First, a nanocomposite material (PEG-carbon black mixed solution) was prepared by mixing a carbon black and polyethylene glycol (PEG 4000 manufactured by Aldrich Chemical Co.) together in deionized water such that the carbon black and the polyethylene glycol would both have the same concentration of 10 mg/ml.

Next, to inhibit oxidation of the polyethylene glycol with ascorbic acid (manufactured by FUJIFILM Wako Pure Chemical Corporation), ascorbic acid was added at a concentration of 0-10 mg/ml to the nanocomposite material. The nanocomposite material thus prepared was deposited on an Si substrate (n-type substrate capped with an SiO₂ layer having a thickness of 100 nm) on which a pair of Pt electrodes had been formed, thereby fabricating a gas sensor (as a device).

In the gas sensor according to the first example, the sensitive membrane thereof contains 10 mg/ml of carbon black, 10 mg/ml of polyethylene glycol, and 1 mg/ml of ascorbic acid. Thus, the sensitive membrane according to the first example contains 10% by mass of ascorbic acid with respect to the polyethylene glycol.

In the gas sensor according to the second example, the sensitive membrane thereof contains 10 mg/ml of carbon black, 10 mg/ml of polyethylene glycol, and 5 mg/ml of ascorbic acid. Thus, the sensitive membrane according to the second example contains 50% by mass of ascorbic acid with respect to the polyethylene glycol.

In the gas sensor according to the third example, the sensitive membrane thereof contains 10 mg/ml of carbon black, 10 mg/ml of polyethylene glycol, and 10 mg/ml of ascorbic acid. Thus, the sensitive membrane according to the third example contains 100% by mass of ascorbic acid with respect to the polyethylene glycol.

On the other hand, in the gas sensor according to the first comparative example, the sensitive membrane thereof contains no ascorbic acid but contains 10 mg/ml of carbon black and 10 mg/ml of polyethylene glycol. Thus, the sensitive membrane according to the first comparative example contains 0% by mass of ascorbic acid with respect to polyethylene glycol.

In the gas sensor, a pair of Pt electrodes with a Ti adhesive layer was formed on a substrate having dimensions of 30×5 mm² by combining a metallic mask and radio frequency (RF) sputtering process. The gap distance and thickness of the Pt electrodes were 2 mm and 300 nm, respectively. The nanocomposite material including the ascorbic acid was applied as a coating onto the substrate by spin coating process (2000 rpm, 200 s). Next, the gas sensor thus formed was annealed at 120° C. for 24 hours in a vacuum to vaporize the solvent.

The gas sensors according to the first to third examples and the first comparative example were subjected to molecule sensing measurement (i.e., sensor sensitivity measurement) using 2.7 ppm of nonanal at room temperature in the air. In this case, nitrogen (N₂) was used as a carrier gas. The readout voltage was 1 V. The sensing response (sensor sensitivity) was defined to be (R_{g}−R_{N2})/R_{N2}×100%, where R_{g} and R_{N2} are resistance values of the sensor exposed to nonanal and N₂, respectively. To conduct an accelerated aging test, each of the gas sensors thus formed (according to the first to third examples and the first comparative example) was maintained at 120° C. in the air having a relatively humidity (RH) of 0%.

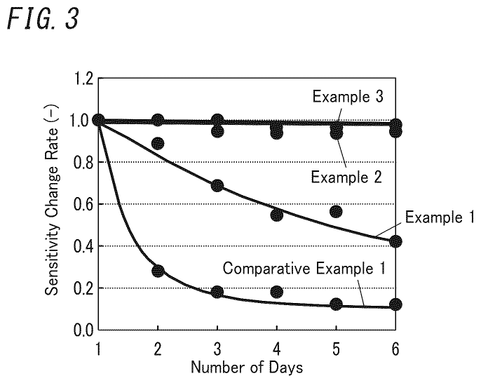

FIG. 3 is a graph showing relationships between the sensor sensitivity change rate and the number of days of the accelerated aging test in the gas sensors according to the first to third examples and the first comparative example. In the first to third examples in which the content of the ascorbic acid is equal to or greater than 10% by mass with respect to polyethylene glycol, even as the number of days of the accelerated aging test increases, the sensor sensitivity decreases less significantly than in the first comparative example. Thus, it can be said that adding 10% by mass or more of an oxidation inhibitor (ascorbic acid) with respect to the sensitive material (polyethylene glycol) effectively reduces the deterioration with time in the sensor sensitivity of the gas sensor.

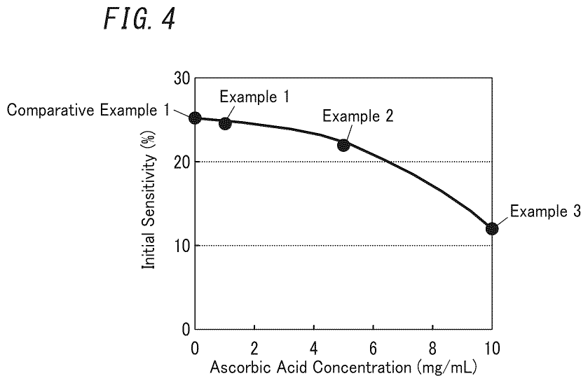

FIG. 4 is a graph showing a relationship between the initial sensitivity of the sensor sensitivity and the content of ascorbic acid with respect to polyethylene glycol in the gas sensors according to the first to third examples and the first comparative example. In the first and second examples in which the content of the ascorbic acid with respect to the polyethylene glycol is equal to or less than 50% by mass, the initial sensor sensitivity decreases less significantly with respect to the first comparative example. Thus, it can be said that adding 50% by mass or less of an oxidation inhibitor (ascorbic acid) with respect to the sensitive material (polyethylene glycol) effectively reduces the chances of causing a decrease in the initial sensor sensitivity of the gas sensor.

### Fourth to Sixth Examples and Second Comparative Example

In fourth to sixth examples and a second comparative example, a gas sensor, including a sensitive membrane made of a polymer-carbon black nanocomposite, was formed.

First, a nanocomposite material (PEG-carbon black mixed solution) was prepared by mixing a carbon black and polyethylene glycol (PEG 4000 manufactured by Aldrich Chemical Co.) together in N-methyl-2-pyrrolidone (NMP). The carbon black and the polyethylene glycol had concentrations of 20 mg/ml and 10 mg/ml, respectively.

Next, as an aromatic oxidation inhibitor, pentaerythritol tetrakis[3-(3,5-di-tert-butyl-4-hydroxyphenyl) propionate] was added to the nanocomposite material so as to have a concentration of 0-10 mg/ml with respect to the polyethylene glycol. The nanocomposite material thus prepared was deposited on an Si substrate (n-type substrate capped with an SiO₂ layer having a thickness of 100 nm) on which a Pt electrode pattern had been formed, thereby making a gas sensor (as a device) as a 16-channel sensor array.

In the gas sensor according to the fourth example, the sensitive membrane thereof contains 20 mg/ml of carbon black, 10 mg/ml of polyethylene glycol, and 1 mg/ml of pentaerythritol tetrakis[3-(3,5-di-tert-butyl-4-hydroxyphenyl) propionate]. Thus, the sensitive membrane according to the fourth example contains 10% by mass of pentaerythritol tetrakis[3-(3,5-di-tert-butyl-4-hydroxyphenyl) propionate] with respect to the polyethylene glycol.

In the gas sensor according to the fifth example, the sensitive membrane thereof contains 20 mg/ml of carbon black, 10 mg/ml of polyethylene glycol, and 5 mg/ml of pentaerythritol tetrakis[3-(3,5-di-tert-butyl-4-hydroxyphenyl) propionate]. Thus, the sensitive membrane according to the fifth example contains 50% by mass of pentaerythritol tetrakis[3-(3,5-di-tert-butyl-4-hydroxyphenyl) propionate] with respect to the polyethylene glycol.

In the gas sensor according to the sixth example, the sensitive membrane thereof contains 20 mg/ml of carbon black, 10 mg/ml of polyethylene glycol, and 10 mg/ml of pentaerythritol tetrakis[3-(3,5-di-tert-butyl-4-hydroxyphenyl) propionate]. Thus, the sensitive membrane according to the sixth example contains 100% by mass of pentaerythritol tetrakis[3-(3,5-di-tert-butyl-4-hydroxyphenyl) propionate] with respect to the polyethylene glycol.

On the other hand, in the gas sensor according to the second comparative example, the sensitive membrane thereof contains no pentaerythritol tetrakis[3-(3,5-di-tert-butyl-4-hydroxyphenyl) propionate] but contains 10 mg/ml of carbon black and 10 mg/ml of polyethylene glycol. Thus, the sensitive membrane according to the second comparative example contains 0% by mass of pentaerythritol tetrakis[3-(3,5-di-tert-butyl-4-hydroxyphenyl) propionate] with respect to the polyethylene glycol.

In the gas sensor, a comb-shaped Pt electrode with a Ti adhesive layer was formed by patterning on a substrate having dimensions of 7×7 mm² by combining photolithography and radio frequency (RF) sputtering process. The gap distance and thickness of the Pt electrode were 40 μm and 400 nm, respectively. Next, an SU-8 photoresist layer with a thickness of 45 μm was formed as a coating by spin coating technique on an electrode patterned substrate and a circular hole was formed by patterning through the SU-8 layer by photolithographic process. The nanocomposite material including the pentaerythritol tetrakis[3-(3,5-di-tert-butyl-4-hydroxyphenyl) propionate] was dripped onto the patterned substrate by an inkjet process. Next, the gas sensor thus formed was annealed at 140° C. for 6 hours in a nitrogen gas atmosphere to vaporize the solvent.

The gas sensors according to the fourth to sixth examples and the second comparative example were subjected to molecule sensing measurement (i.e., sensor sensitivity measurement) using 10 ppm of benzaldehyde at room temperature in the air. In this case, nitrogen (N₂) was used as a carrier gas. The readout voltage was 1 V. The sensing response (sensor sensitivity) was defined to be (R_{g}−R_{N2})/R_{N2×100}%, where R_{g} and R_{N2} are resistance values of the sensor exposed to benzaldehyde and N₂, respectively. To conduct an accelerated aging test, each of the gas sensors thus formed (according to the fourth to sixth examples and the second comparative example) was maintained at 85° C. for 90 hours in the air having a relatively humidity (RH) of 85%.

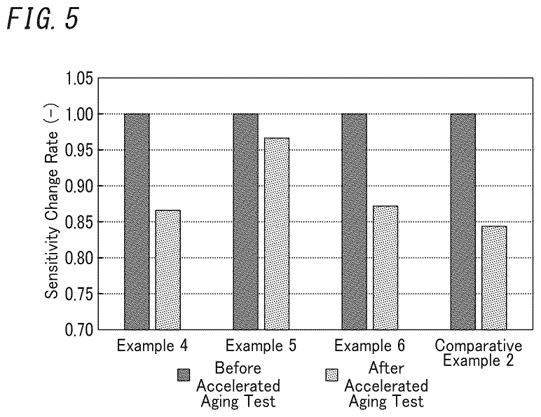

FIG. 5 is a graph showing, in comparison, respective change rates of the sensor sensitivity after the accelerated aging test to the sensor sensitivity before the accelerated aging test in the gas sensors according to the fourth to sixth examples and the second comparative example. In the fourth to sixth examples in which the content of pentaerythritol tetrakis[3-(3,5-di-tert-butyl-4-hydroxyphenyl) propionate] with respect to the polyethylene glycol is equal to or greater than 10% by mass, the sensor sensitivity decreases after the accelerated aging test less significantly than in the second comparative example. Thus, it can be said that adding 10% by mass or more of an aromatic oxidation inhibitor (pentaerythritol tetrakis[3-(3,5-di-tert-butyl-4-hydroxyphenyl) propionate]) to the sensitive material (polyethylene glycol) effectively reduces the deterioration with time in the sensor sensitivity of the gas sensor in a high humidity environment.

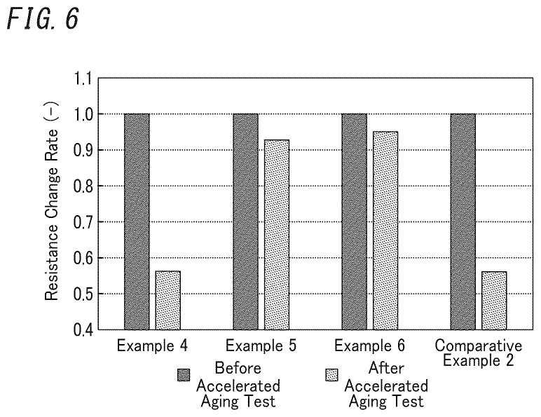

FIG. 6 is a graph showing, in comparison, the respective change rates of the sensor resistance (R_{N2}) after the accelerated aging test to the sensor resistance (R_{N2}) before the accelerated aging test in the gas sensors according to the fourth to sixth examples and the second comparative example. In the fifth and sixth examples in which the content of pentaerythritol tetrakis[3-(3,5-di-tert-butyl-4-hydroxyphenyl) propionate] with respect to the polyethylene glycol is equal to or greater than 50% by mass, the sensor resistance (R_{N2}) changes after the accelerated aging test less significantly than in the second comparative example. Thus, it can be said that adding 50% by mass or more of an aromatic oxidation inhibitor (pentaerythritol tetrakis[3-(3,5-di-tert-butyl-4-hydroxyphenyl) propionate]) to the sensitive material (polyethylene glycol) effectively reduces the change with time in the sensor resistance (R_{N2}) of the gas sensor in a high humidity environment.

### Seventh Example and Third Comparative Example

In a seventh example and a third comparative example, a gas sensor, including a sensitive membrane made of a polymer-carbon black nanocomposite, was formed.

First, a nanocomposite material (PEG-carbon black mixed solution) was prepared by mixing a carbon black and polyethylene glycol (PEG 4000 manufactured by Aldrich Chemical Co.) together in deionized water such that the carbon black and the polyethylene glycol would both have the same concentration of 10 mg/ml.

Next, to inhibit oxidation of the polyethylene glycol with ascorbic acid (manufactured by FUJIFILM Wako Pure Chemical Corporation), ascorbic acid was added at a concentration of 0-5 mg/ml to the nanocomposite material. The nanocomposite material thus prepared was deposited on an Si substrate (n-type substrate capped with an SiO₂ layer having a thickness of 100 nm) on which a Pt electrode pattern had been formed, thereby fabricating a gas sensor (as a device) as a 16-channel sensor array.

In the gas sensor according to the seventh example, the sensitive membrane thereof contains 10 mg/ml of carbon black, 10 mg/ml of polyethylene glycol, and 5 mg/ml of ascorbic acid. Thus, the sensitive membrane according to the seventh example contains 50% by mass of ascorbic acid with respect to the polyethylene glycol.

On the other hand, in the gas sensor according to the third comparative example, the sensitive membrane thereof contains no ascorbic acid but contains 10 mg/ml of carbon black and 10 mg/ml of polyethylene glycol. Thus, the sensitive membrane according to the third comparative example contains 0% by mass of ascorbic acid with respect to the polyethylene glycol.

In the gas sensor, a comb-shaped Pt electrode with a Ti adhesive layer was formed by patterning on a substrate having dimensions of 7×7 mm² by combining photolithography and radio frequency (RF) sputtering process. The gap distance and thickness of the Pt electrode were 40 μm and 400 nm, respectively. Next, an SU-8 photoresist layer with a thickness of 45 μm was formed as a coating by spin coating technique on an electrode patterned substrate and a circular hole was formed by patterning through the SU-8 layer by photolithographic process. The nanocomposite material including the ascorbic acid was dripped onto the patterned substrate by an inkjet process. Next, the gas sensor thus fabricated was annealed at 120° C. for 24 hours in a vacuum to vaporize the solvent.

The gas sensors according to the seventh example and the third comparative example were subjected to molecule sensing measurement (i.e., sensor sensitivity measurement) using 2.7 ppm of nonanal at room temperature in the air. In this case, nitrogen (N₂) was used as a carrier gas. The readout voltage was 1 V. The sensing response (sensor sensitivity) was defined to be (R_{g}−R_{N2})/R_{N2}×100%, where R_{g} and R_{N2} are resistance values of the sensor exposed to nonanal and N₂, respectively. To estimate the decrease in sensitivity within the air atmosphere, the gas sensors thus fabricated (according to the seventh example and the third comparative example) were stored in the air with a relatively humidity (RH) of 67%.

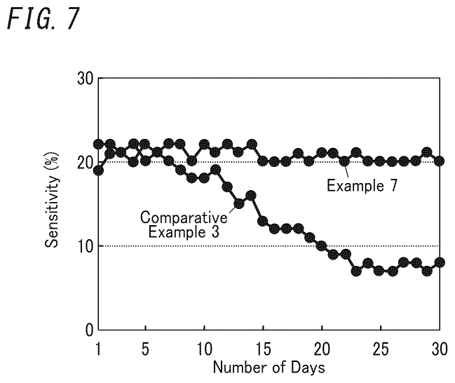

FIG. 7 is a graph showing relationships between the sensor sensitivity change rate and the number of days of storage in the air in the gas sensors according to the seventh example and the third comparative example. In the seventh example in which the content of the ascorbic acid is 50% by mass with respect to the polyethylene glycol, the sensor sensitivity decreases less significantly than in the third comparative example even as the number of days of storage increases. This result reveals that adding 50% by mass of ascorbic acid would reduce the chances of causing not only a decrease in sensor sensitivity due to the oxidation of the sensitive membrane but also a decrease in sensor sensitivity due to adsorption of moisture into the sensitive membrane. Thus, it can be said that adding 50% by mass of oxidation inhibitor (ascorbic acid) with respect to the sensitive material (polyethylene glycol) in the gas sensor having the above-described configuration effectively reduces the deterioration with time in the sensor sensitivity of the gas sensor.

### REFERENCE SIGNS LIST

1 Gas Sensor
20 Sensitive Membrane
21 Electrode
201 Membrane Body

1. A sensitive membrane comprising:
a membrane body containing a sensitive material;
a conductive material contained in the membrane body; and
an oxidation inhibitor,
the oxidation inhibitor being contained in the membrane body to inhibit oxidation of the sensitive material.

2. The sensitive membrane of claim 1, wherein
the sensitive material includes an organic polymer.

3. The sensitive membrane of claim 1, wherein
the sensitive material includes an ionic liquid.

4. The sensitive membrane of claim 1, wherein
the conductive material includes at least one selected from the group consisting of carbon blacks, carbon nanotubes, metallic nanoparticles, and conductive polymers.

5. The sensitive membrane of claim 2, wherein
the organic polymer includes at least one selected from the group consisting of polyethers, polyesters, and silicones.

6. The sensitive membrane of claim 1, wherein
the oxidation inhibitor includes at least one selected from the group consisting of aromatic compounds, sulfur compounds, phosphorus compounds, amine compounds, metallic compounds, vitamin E, and vitamin C.

7. The sensitive membrane of claim 1, wherein
content of the oxidation inhibitor is equal to or greater than 10% by mass and equal to or less than 50% by mass with respect to the sensitive material.

8. A gas sensor comprising:
the sensitive membrane of claim 1; and
an electrode electrically connected to the conductive material.
